(19) United States
(12) Patent Application Publication
(10) Pub. No.: US 2010/0222381 A1
(43) Pub. Date: Sep. 2, 2010

(54) Cyclopentathiophene/cyclohexathiophene DNA methyltransferase inhibitors

(76) Inventors: Hariprasad Vankayalapati, Draper, UT (US); Krzysztof Swierczek, West Jordan, UT (US); Scott Albert Pearce, Clearfield, UT (US)

Correspondence Address: SuperGen, Inc., 4140 Dublin Blvd., Suite 200, Dublin, CA, 94568, US

(21) Appl. No.: 12/660,477

(22) Filed: Feb. 26, 2010

Related U.S. Application Data
(60) Provisional application No. 61/208,772, filed on Feb. 27, 2009.

(51) Int. Cl. A61K 31/4709; C07D 209/30; C07D 405/12; C07D 333/04; C07D 215/20; A61K 31/404; A61K 31/4025; A61K 31/381; A61P 35/00

(52) U.S. Cl. 514/312; 548/465; 548/525; 549/57; 546/153; 514/414; 514/422; 514/443

(57) ABSTRACT

Compounds represented by Formula (I):

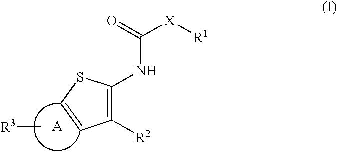

are useful in treating diseases, such as cancer, that are mediated and/or associated (at least in part) with DNMT3b activity. The compounds can be formulated as pharmaceutically acceptable compositions for administration to a subject in need thereof.

This application claims the benefit of U.S. Patent Application 61/208,772 filed 27 Feb. 2009.

### FIELD OF THE INVENTION

The present invention relates generally to cyclohexathiphene fused 5,6 and cyclopentathiophene 5,5 hetero ring compounds that inhibit DNA methyltransferase activity—including DNA methyltransferase 3 beta (DNMT3b) activity, and to compositions and methods related thereto. In particular, the present invention relates to 4,5,6,7-tetrahydrobenzo[b]thiophenyl and 5,6-dihydro-4H-cyclopenta[b]thiophenyl compounds that inhibit DNMT3b activity, useful in the treatment of cancer and hyperproliferative diseases.

### DESCRIPTION OF THE RELATED ART

Cancer (and other hyperproliferative diseases) is characterized by uncontrolled cell proliferation. This loss of the normal control of cell proliferation often appears as the result of genetic damage to cell pathways that control progress through the cell cycle. Such change includes resulting abnormal methylation patterns in malignant cells. Elevated levels of DNA methyltransferases, of which DNMT3b is one, in tumors contribute to tumorigenesis by improper de novo methylation and silencing of promoters for growth-regulating genes. Inhibition of the DNMT function, particularly DNMT3b that is especially involved in de novo methylation, would lead to new compounds useful in the treatment of cancer.

Based on the involvement in a number of human malignancies, there is a need for the design of specific and selective inhibitors for the treatment of cancer and other conditions mediated and/or associated with DNMT3b. The present invention fulfills these needs and offers other related advantages.

International Patent Publication No. WO 2008150899 describes Nf-kB inhibitor-p38 MAP kinase inhibitor combination for the treatment of cancer and inflammatory diseases. International Patent Publication No. WO 2008145398 describes preparation of 4-arylpyrrole substituted 2-indoline derivatives as protein kinase inhibitors. U.S. Patent Publication No. US20080269234 describes preparation of fused pyridazine derivatives as inhibitors of poly(ADP-ribose)polymerase. International Patent Publication No. WO 2008125111 describes preparation of triazolopyridines as phosphodiesterase inhibitors for treatment of dermal diseases. International Patent Publication No. WO 2008124083 describes phtalazonamine derivatives and related compounds as aurora kinase modulators. International Patent Publication No. WO 2008115999 describes biaryl and biheteroaryl compounds useful in iron disorders. International Patent Publication No. WO 2008106202 describes theramutein modulators. U.S. Patent Publication No. US20080194650 describes preparation of aryl fluoroethyl ureas as therapeutic alpha2 adrenergic agents. International Patent Publication No. WO 2008094992 describes 2-aminopyridine-3-carboxamides. International Patent Publication No. WO 2008079719 describes preparation of pyrimidines as aurora kinase inhibitors. U.S. Patent Publication No. US20080188500 describes preparation of [(pyrimidoindolyl)phenyl]benzamide derivatives and analogs. U.S. Patent Publication No. US20080161280 describes preparation of fused pyridazine derivatives.

International Patent Publication No. WO 2008073306 describes preparation of 3-amino-2-oxo-1,2-dihydropyridine amino acid derivatives. International Patent Publication No. WO 2008073305 describes preparation of 2-amino-3-oxo-3,4-dihydropyrazine and 5-amino-4-oxo-3,4-dihydropyrimidine amino acid derivatives. International Patent Publication No. WO 2008067644 describes preparation of pyrazolylbenzimidazole derivatives. International Patent Publication No. WO 2008063300 describes preparation of arylboonates. International Patent Publication No. WO 2008056259 describes preparation of oxazole derivatives. International Patent Publication No. WO 2008020227 describes preparation of pyrrolylcarbonylaminohexahydroazepanylthiozolecarboxylates. International Patent Publication No. WO 2008006583 describes preparation of pyrimidine derivatives. International Patent Publication No. WO 2007135036 describes process of preparation of chiral cyclic beta-aminocarboxamides. International Patent Publication No. WO 2007128460 describes preparation of 3-amino-4-hydroxy pyrrolidine derivatives. International Patent Publication No. WO 2007087717 describes preparation of carboxamidoaryl carboxylic acids. U.S. Patent Publication No. US20070232627 describes preparation of naphthyridines and pyridopyrimidines. U.S. Patent Publication No. US20070232645 describes preparation of 1,6- and 1,8-naphthyridines.

International Patent Publication No. WO 2007068422 describes 1,2,4-triazinolidine-3-thione derivatives. International Patent Publication No. WO 2006123257 describes pyrrole-substituted oxadiazole derivatives. International Patent Publication Nos. WO 2006123255 and WO 2006123249 describe novel oxadiazole derivatives. International Patent Publication No. WO 2006123244 describes carbamate deriviatives. International Patent Publication No. WO 200696444 describes preparation of heteroarylmethyl substituted octahydro-1,10-phenanthrolines. U.S. Patent Publication No. US20060160812 describes methods for treating neural disorders and heterocyclic compounds useful therefor. U.S. Patent Publication No. US20060189628 describes preparation of piperidinyl- and (homo)piperazinylpyrrolidinols. U.S. Patent Publication No. US20080125432 describes preparation of 5-carboxamido thiazoles. U.S. Patent Publication No. US20070299110 describes preparation of novel tetrazole derivatives. U.S. Patent Publication No. US20080045537 describes preparation of (quinolinylaminoalkyl)-benzimidazole derivatives. U.S. Patent Publication No. US20060019967 describes SARS cov main protease inhibitors. U.S. Patent Publication No. US20080009488 describes preparation of Raf modulators.

U.S. Patent Publication No. US20050250789 describes preparation of N-heterocyclic hydroxamic acid derivatives. U.S. Patent Publication No. US20080004263 describes preparation of isoxazolylthiazoles. Japanese Patent Publication No. JP2005162720 describes sugar metabolism-improving agents containing endothelial differentiation gene 1 agonist. International Patent Publication No. WO 2005048948 describes urea derivatives. International Patent Publication No. WO 2005048953 describes amide derivatives. International Patent Publication No. WO 2005044797 describes preparation of piperidine derivatives. U.S. Patent Publication No. US20050256161 describes amine-containing phenyl derivatives. U.S. Patent Publication No. US20060211677 describes preparation of diphenyl substituted cycloalkanes. U.S. Pat. No. 7,037,909 describes tetracyclic compounds, namely 9H-1,2,3a,4,9,10-hexaazacyclopenta[b]fluorine derivatives and analogs. U.S. Patent Publication No. US20060160812 describes methods for treating neural disorders and heterocyclic compounds useful therefor. U.S. Pat. No. 7,223,780 describes preparation of triazole- and tetrazolecarboxamides. U.S. Pat. No. 7,317,027 describes preparation of thienylisoxazolylmethylazaindoles.

U.S. Patent Publication No. 20040186148 describes preparation of benzene derivatives. U.S. Patent Publication No. US20050267114 describes preparation of triazaspiro[5.5]undecane derivatives. U.S. Patent Publication No. US20030073832 describes preparation of aminophenyl(hetero)aryl ketones. U.S. Pat. No. 6,784,185 describes preparation of pyrazolo[4,3-d]pyrimidinones. U.S. Pat. No. 7,157,487 describes preparation of chiral pyrrolidine derivatives. U.S. Pat. No. 7,435,747 describes preparation of guanidines and amidines. U.S. Pat. No. 6,849,660 describes antimicrobial biaryl compounds. U.S. Pat. No. 6,486,142 describes phosphonic acid derivatives. U.S. Pat. Nos. 6,310,060, 6,506,789, 6,492,363, and 7019033 describe preparation of 2-(4-bromo or 4-iodo phenylamino)benzoic acid derivatives.

The following compounds are known from various chemical libraries:

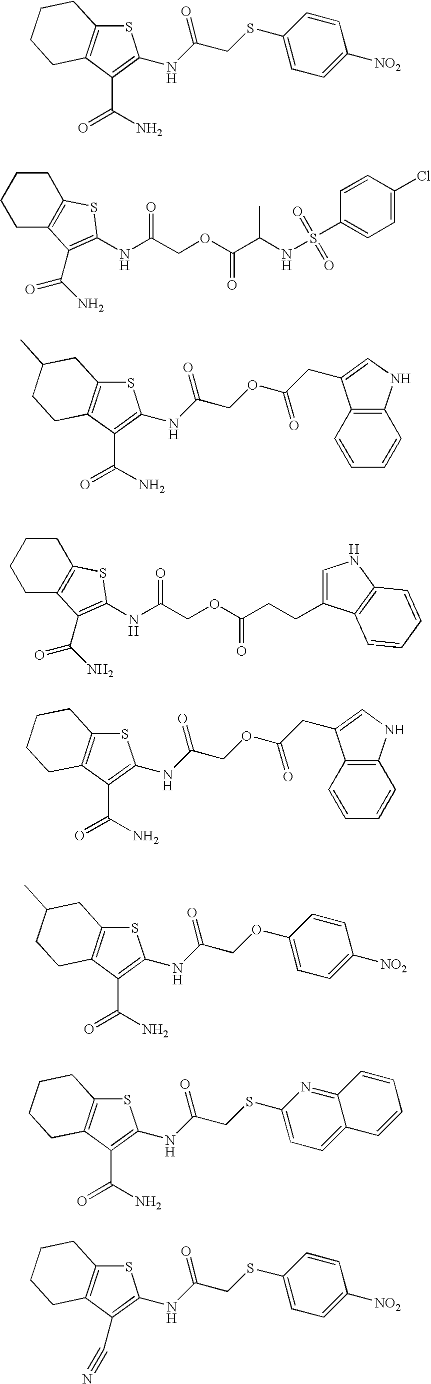

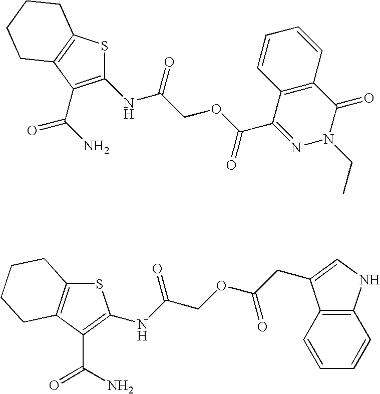

### BRIEF SUMMARY OF THE INVENTION

The present invention is generally directed to compounds having the following general Formula (I):

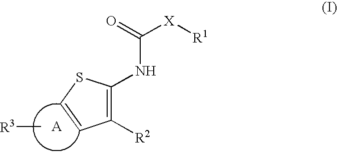

useful in treating diseases, such as cancer, that are mediated and/or associated (at least in part) with DNMT3b activity. The compounds can be formulated as pharmaceutically acceptable compositions for administration to a subject in need thereof.

These and other aspects of the invention will be apparent upon reference to the following detailed description. To that end, certain patent and other documents are cited herein to more specifically set forth various aspects of this invention. Each of these documents is hereby incorporated by reference in its entirety.

### DETAILED DESCRIPTION OF THE INVENTION

The present invention is generally directed to compounds having the following general structure according to Formula (I):

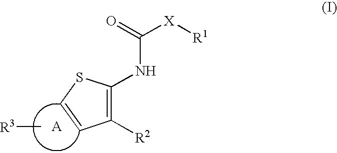

and pharmaceutically acceptable salts thereof, wherein:

A is cyclopentenyl or cyclohexenyl;

X is —CH₂—O—, —CH₂—S—, —CH(CH₃)—O—, —CH(CH₃)—S—, -furanyl-CH₂—, or a direct bond;

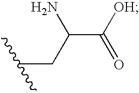

R¹ is aryl, heteroaryl, heterocyclyl, or each optionally substituted with 1-3 independent C₁₋₄alkyl, NO₂, COOH, or —NH(C₀₋₄alkyl)-aryl substituents;

R² is H, —C(O)—NH₂, or COOH; and

R³ is C₀₋₄alkyl;

provided that the compound is not:

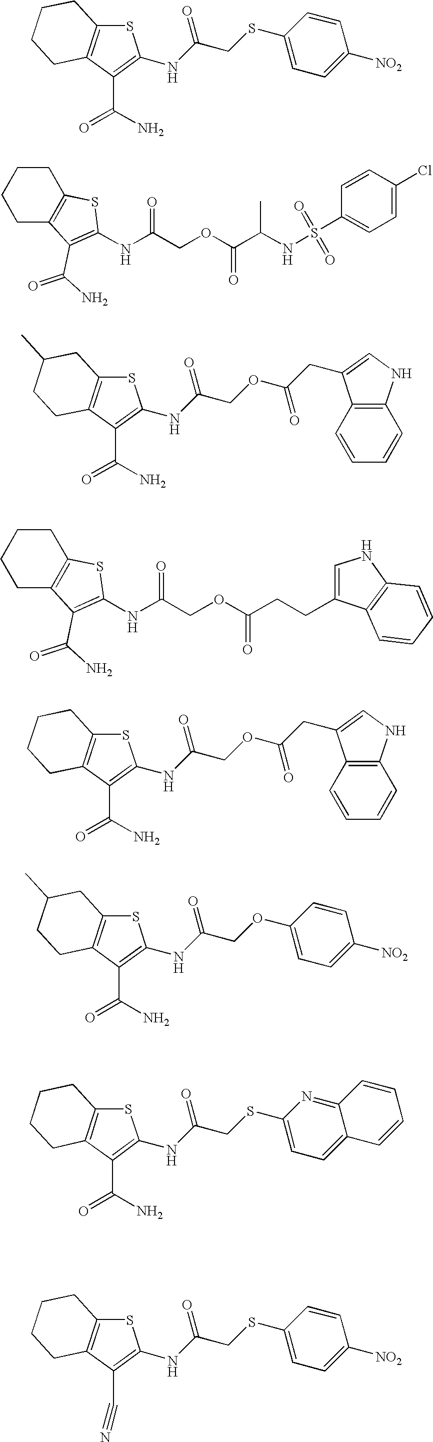

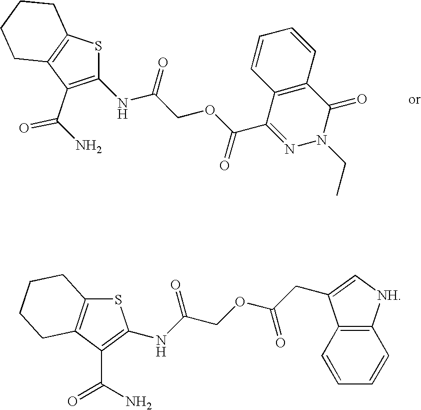

In an aspect of the invention, compounds of the present invention are described by Formula (I) and pharmaceutically acceptable salts thereof, wherein A is cyclopentenyl and the other variables are as defined above for Formula (I).

In an embodiment of this aspect, compounds of the present invention are described by Formula (I) and pharmaceutically acceptable salts thereof, wherein A is cyclopentenyl, X is —CH₂—O—, and the other variables are as defined above for Formula (I).

In another embodiment of this aspect, compounds of the present invention are described by Formula (I) and pharmaceutically acceptable salts thereof, wherein A is cyclopentenyl, X is —CH₂—O—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclopentenyl, X is —CH₂—O—, R¹ is heterocyclyl optionally substituted with 1-independent C₁₋₄alkyl, NO₂, COOH, or —NH(C₀₋₄-alkyl)-aryl substituents, and the other variables are as defined above for Formula (I).

In still another embodiment of this aspect, compounds of the present invention are described by Formula (I) and pharmaceutically acceptable salts thereof, wherein A is cyclopentenyl, X is —CH₂—O—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclopentenyl, X is —CH₂—S—, and the other variables are as defined above for Formula (I).

In another embodiment of this aspect, compounds of the present invention are described by Formula (I) and pharmaceutically acceptable salts thereof, wherein A is cyclopentenyl, X is —CH₂—S—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclopentenyl, X is —CH₂—S—, R¹ is heterocyclyl optionally substituted with 1-3 independent C₁₋₄alkyl, NO₂, COOH, or —NH(C₀₋₄-alkyl)-aryl substituents, and the other variables are as defined above for Formula (I).

In still another embodiment of this aspect, compounds of the present invention are described by Formula (I) and pharmaceutically acceptable salts thereof, wherein A is cyclopentenyl, X is —CH₂—S—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclopentenyl, X is —CH(CH₃)—O—, and the other variables are as defined above for Formula (I).

In another embodiment of this aspect, compounds of the present invention are described by Formula (I) and pharmaceutically acceptable salts thereof, wherein A is cyclopentenyl, X is —CH(CH₃)—O—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclopentenyl, X is —CH(CH₃)—O—, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclopentenyl, X is —CH(CH₃)—O—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclopentenyl, X is —CH(CH₃)—S—, and the other variables are as defined above for Formula (I).

In another embodiment of this aspect, compounds of the present invention are described by Formula (I) and pharmaceutically acceptable salts thereof, wherein A is cyclopentenyl, X is —CH(CH₃)—S—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclopentenyl, X is —CH(CH₃)—S—, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclopentenyl, X is —CH(CH₃)—S—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclopentenyl, X is -furanyl-CH₂—, and the other variables are as defined above for Formula (I).

In another embodiment of this aspect, compounds of the present invention are described by Formula (I) and pharmaceutically acceptable salts thereof, wherein A is cyclopentenyl, X is -furanyl-CH₂—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclopentenyl, X is -furanyl-CH₂—, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclopentenyl, X is -furanyl-CH₂—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclopentenyl, X is a direct bond, and the other variables are as defined above for Formula (I).

In another embodiment of this aspect, compounds of the present invention are described by Formula (I) and pharmaceutically acceptable salts thereof, wherein A is cyclopentenyl, X is a direct bond, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclopentenyl, X is a direct bond, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclopentenyl, X is a direct bond, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an aspect of the invention, compounds of the present invention are described by Formula (I) and pharmaceutically acceptable salts thereof, wherein A is cyclohexenyl and the other variables are as defined above for Formula (I).

In an embodiment of this aspect, compounds of the present invention are described by Formula (I) and pharmaceutically acceptable salts thereof, wherein A is cyclohexenyl, X is —CH₂—O—, and the other variables are as defined above for Formula (I).

In another embodiment of this aspect, compounds of the present invention are described by Formula (I) and pharmaceutically acceptable salts thereof, wherein A is cyclohexenyl, X is —CH₂—O—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclohexenyl, X is —CH₂—O—, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclohexenyl, X is —CH₂—O—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclohexenyl, X is —CH₂—S—, and the other variables are as defined above for Formula (I).

In another embodiment of this aspect, compounds of the present invention are described by Formula (I) and pharmaceutically acceptable salts thereof, wherein A is cyclohexenyl, X is —CH₂—S—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclohexenyl, X is —CH₂—S—, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclohexenyl, X is —CH₂—S—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclohexenyl, X is —CH(CH₃)—O—, and the other variables are as defined above for Formula (I). In another embodiment of this aspect, compounds of the present invention are described by Formula (I) and pharmaceutically acceptable salts thereof, wherein A is cyclohexenyl, X is —CH(CH₃)—O—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclohexenyl, X is —CH(CH₃)—O—, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclohexenyl, X is —CH(CH₃)—O—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclohexenyl, X is —CH(CH₃)—S—, and the other variables are as defined above for Formula (I).

In another embodiment of this aspect, compounds of the present invention are described by Formula (I) and pharmaceutically acceptable salts thereof, wherein A is cyclohexenyl, X is —CH(CH₃)—S—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclohexenyl, X is —CH(CH₃)—S—, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclohexenyl, X is —CH(CH₃)—S—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclohexenyl, X is -furanyl-CH₂—, and the other variables are as defined above for Formula (I).

In another embodiment of this aspect, compounds of the present invention are described by Formula (I) and pharmaceutically acceptable salts thereof, wherein A is cyclohexenyl, X is -furanyl-CH₂—, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclohexenyl, X is -furanyl-CH₂—, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclohexenyl, X is -furanyl-CH₂—, R¹ is heteroaryl optionally substituted with 1-3 independent C₁₋₄alkyl, NO₂, COOH, or —NH(C₀₋₄alkyl)-aryl substituents, and the other variables are as defined above for Formula (I).

In an embodiment of this aspect, compounds of the present invention are described by Formula (I) and pharmaceutically acceptable salts thereof, wherein A is cyclohexenyl, X is a direct bond, and the other variables are as defined above for Formula (I).

In another embodiment of this aspect, compounds of the present invention are described by Formula (I) and pharmaceutically acceptable salts thereof, wherein A is cyclohexenyl, X is a direct bond, R¹ is aryl optionally substituted with 1-3 independent C₁₋₄alkyl, NO₂, COOH, or —NH(C₀₋₄alkyl)-aryl substituents, and the other variables are as defined above for Formula (I).

In yet another embodiment of this aspect, compounds of the present invention are described by Formula (I) and pharmaceutically acceptable salts thereof, wherein A is cyclohexenyl, X is a direct bond, R¹ is heterocyclyl optionally substituted with 1-3 independent C₁₋₄alkyl, NO₂, COOH, or —NH(C₀₋₄alkyl)-aryl substituents, and the other variables are as defined above for Formula (I).

In still another embodiment of this aspect, compounds of the present invention are described by Formula (I) and pharmaceutically acceptable salts thereof, wherein A is cyclohexenyl, X is a direct bond, R¹ is heteroaryl optionally substituted with 1-3 independent C₁₋₄alkyl, NO₂, COOH, or —NH(C₀₋₄alkyl)-aryl substituents, and the other variables are as defined above for Formula (I). These compounds have utility over a broad range of therapeutic applications, and may be used to treat diseases, such as cancer, that are mediated and/or associated (at least in part) with DNMT3b activity. Accordingly, in one aspect of the invention, the compounds described herein are formulated as pharmaceutically acceptable compositions for administration to a subject in need thereof.

In another aspect, the invention provides methods for treating or preventing a DNMT3b activity-mediated disease, such as cancer, which method comprises administering to a patient in need of such a treatment a therapeutically effective amount of a compound described herein or a pharmaceutically acceptable composition comprising said compound.

Another aspect relates to inhibiting DNMT3b activity in a biological sample, which method comprises contacting the biological sample with a compound described herein, or a pharmaceutically acceptable composition comprising said compound.

Another aspect relates to a method of inhibiting DNMT3b activity in a patient, which method comprises administering to the patient a compound described herein or a pharmaceutically acceptable composition comprising said compound.

These and other aspects of the invention will be apparent upon reference to the following detailed description. To that end, certain patent and other documents are cited herein to more specifically set forth various aspects of this invention. Each of these documents is hereby incorporated by reference in its entirety.

Unless otherwise stated the following terms used in the specification and claims have the meanings discussed below:

“Alkyl” refers to a saturated straight or branched hydrocarbon radical of one to six carbon atoms, preferably one to four carbon atoms, e.g., methyl, ethyl, propyl, 2-propyl, n-butyl, iso-butyl, tert-butyl, pentyl, hexyl, and the like, preferably methyl, ethyl, propyl, or 2-propyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Cyclic alkyls are referred to herein as a “cycloalkyl.”

Unsaturated alkyls contain at least one double or triple bond between adjacent carbon atoms (referred to as an “alkenyl” or “alkynyl”, respectively.) Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“C₀₋₄alkyl” refers to an alkyl with 0, 1, 2, 3, or 4 carbon atoms. C₀₋₄alkyl with 0 carbon atoms is a hydrogen atom when terminal and is a direct bond when linking.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like, preferably methylene, ethylene, or propylene.

“Cycloalkyl” refers to a saturated cyclic hydrocarbon radical of three to eight carbon atoms, e.g., cyclopropyl, cyclobutyl, cyclopentyl or cyclohexyl.

“Alkoxy” means a radical —ORₐ where Rₐ is an alkyl as defined above, e.g., methoxy, ethoxy, propoxy, butoxy and the like.

“Halo” means fluoro, chloro, bromo, or iodo, preferably fluoro and chloro.

(77) “Haloalkyl” means alkyl substituted with one or more, preferably one, two or three, same or different halo atoms, e.g., —CH₂Cl, —CF₃, —CH₂CF₃, —CH₂CCl₃, and the like.

“Haloalkoxy” means a radical —OR_{b} where R_{b} is an haloalkyl as defined above, e.g., trifluoromethoxy, trichloroethoxy, 2,2-dichloropropoxy, and the like.

“Acyl” means a radical —C(O)R_{c}, where R_{c} is hydrogen, alkyl, or haloalkyl as defined herein, e.g., formyl, acetyl, trifluoroacetyl, butanoyl, and the like.

“Aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The aryl group may be substituted or unsubstituted. Unless specifically stated otherwise, “substituted aryl” refers to the aryl group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, phenoxy, heteroaryl, heteroaryloxy, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heteroaryl” refers to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, and carbazole. The heteroaryl group may be unsubstituted or substituted, such as, for example, 5-methylthiazolyl. Unless specifically stated otherwise, “substituted heteroaryl” refers to the heteroaryl group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Carbocycle” refers to a saturated, unsaturated or aromatic ring system having 3 to 14 ring carbon atoms. The term “carbocycle”, whether saturated or partially unsaturated, also refers to rings that are optionally substituted. The term “carbocycle” includes aryl. The term “carbocycle” also includes aliphatic rings that are fused to one or more aromatic or nonaromatic rings, such as in a decahydronaphthyl or tetrahydronaphthyl, where the radical or point of attachment is on the aliphatic ring. The carbocycle group may be substituted or unsubstituted. Unless specifically stated otherwise, “substituted carbocycle” refers to the carbocycle group being substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heterocycle” refers to a saturated, unsaturated or aromatic cyclic ring system having 3 to 14 ring atoms in which one, two or three ring atoms are heteroatoms selected from N, O, or S(O)ₘ (where m is an integer from 0 to 2), the remaining ring atoms being C, where one or two C atoms may optionally be replaced by a carbonyl group. The term “heterocycle” includes heteroaryl. Unless specifically stated otherwise, “substituted heterocyclyl” refers to the heterocyclyl ring being substituted independently with one or more, preferably one, two, or three substituents selected from alkyl (wherein the alkyl may be optionally substituted with one or two substituents), haloalkyl, cycloalkylamino, cycloalkylalkyl, cycloalkylaminoalkyl, cycloalkylalkylaminoalkyl, cyanoalkyl, halo, nitro, cyano, hydroxy, alkoxy, amino, alkylamino, dialkylamino, hydroxyalkyl, carboxyalkyl, aminoalkyl, alkylaminoalkyl, dialkylaminoalkyl, aralkyl, heteroaralkyl, aryl, heteroaryl, carbocycle, heterocycle (wherein the aryl, heteroaryl, carbocycle or heterocycle may be optionally substituted), aralkyl, heteroaralkyl, saturated or unsaturated heterocycloamino, saturated or unsaturated heterocycloaminoalkyl, and —COR_{d} (where R_{d} is alkyl). More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin-3-yl, pyrrolidino, morpholino, 4-cyclopropylmethylpiperazino, thiomorpholino, thiomorpholino-1-oxide, thiomorpholino-1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof, including 2-methyl-4,5,6,7-tetrahydro-1H-pyrrolo[2,3-c]pyridinyl. In certain embodiments, the heterocycle group is optionally substituted with one or two substituents independently selected from halo, alkyl, alkyl substituted with carboxy, ester, hydroxy, alkylamino, saturated or unsaturated heterocycloamino, saturated or unsaturated heterocycloaminoalkyl, or dialkylamino.

“Optional” or “optionally” means that the subsequently described event or circumstance may but need not occur, and that the description includes instances where the event or circumstance occurs and instances in which it does not. For example, “heterocyclic group optionally substituted with an alkyl group” means that the alkyl may but need not be present, and the description includes situations where the heterocycle group is substituted with an alkyl group and situations where the heterocycle group is not substituted with the alkyl group.

Lastly, unless specifically stated otherwise, the term “substituted” as used herein means any of the above groups (e.g., alkyl, aryl, heteroaryl, carbocycle, heterocycle, etc.) wherein at least one hydrogen atom is replaced with a substituent. In the case of an oxo substituent (“═O”) two hydrogen atoms are replaced. “Substituents” within the context of this invention include halogen, hydroxy, oxo, cyano, nitro, amino, alkylamino, dialkylamino, alkyl, alkoxy, thioalkyl, haloalkyl (e.g., —CF₃), hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocycloalkyl, substituted heterocycloalkyl, —NRₑR_{f}, —NRₑC(═O)R_{f}, —NRₑ C(═O)NRₑR_{f}, —NRₑC(═O)OR_{f}—NRₑSO₂R_{f}, —ORₑ, —C(═O)Rₑ—C(═O)ORₑ, —C(═O)NRₑR_{f}, —OC(═O)NRₑR_{f}, —SH, —SRₑ, —SORₑ, —S(═O)₂Rₑ, —OS(═O)₂Rₑ, —S(═O)₂ORₑ, wherein Rₑ and R_{f} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocycloalkyl or substituted heterocycloalkyl.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog (Cahn, R., Ingold, C., and Prelog, V. Angew. Chem. 78:413-47, 1966; Angew. Chem. Internat. Ed. Eng. 5:385-415, 511, 1966), or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Ch. 4 of ADVANCED ORGANIC CHEMISTRY, 4^{th} edition, March, J., John Wiley and Sons, New York City, 1992).

The compounds of the present invention may exhibit the phenomena of tautomerism and structural isomerism. This invention encompasses any tautomeric or structural isomeric form and mixtures thereof which possess the ability to modulate DNMT3b activity and is not limited to, any one tautomeric or structural isomeric form.

It is contemplated that a compound of the present invention would be metabolized by enzymes in the body of the organism such as human being to generate a metabolite that can modulate the activity of the protein kinases. Such metabolites are within the scope of the present invention.

A compound of the present invention or a pharmaceutically acceptable salt thereof, can be administered as such to a human patient or can be administered in pharmaceutical compositions in which the foregoing materials are mixed with suitable carriers or excipient(s). Techniques for formulation and administration of drugs may be found, for example, in REMINGTON'S PHARMACOLOGICAL SCIENCES, Mack Publishing Co., Easton, Pa., latest edition.

A “pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

“Pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Pharmaceutically acceptable salt” refers to those salts which retain the biological effectiveness and properties of the parent compound. Such salts may include: (1) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D)- or (L)-malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The compound of the present invention may also act, or be designed to act, as a prodrug. A “prodrug” refers to an agent, which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention, which is, administered as an ester (the “prodrug”), phosphate, amide, carbamate, or urea.

“Therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

The term “protein kinase-mediated condition” or “disease”, as used herein, means any disease or other deleterious condition in which a protein kinase is known to play a role. The term “protein kinase-mediated condition” or “disease” also means those diseases or conditions that are alleviated by treatment with a protein kinase inhibitor. Such conditions include, without limitation, cancer and other hyperproliferative disorders. In certain embodiments, the cancer is a cancer of colon, breast, stomach, prostate, pancreas, or ovarian tissue.

The term “DNMT3b activity-mediated condition” or “disease”, as used herein, means any disease or other deleterious condition in which DNMT3b activity is known to play a role. The term “DNMT3b activity-mediated condition” also means those diseases or conditions that are alleviated by treatment with a DNMT3b inhibitor.

As used herein, “administer” or “administration” refers to the delivery of an inventive compound or of a pharmaceutically acceptable salt thereof or of a pharmaceutical composition containing an inventive compound or a pharmaceutically acceptable salt thereof of this invention to an organism for the purpose of prevention or treatment of a protein kinase-related disorder.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. In certain embodiments, the preferred routes of administration are oral and intravenous. Alternatively, one may administer the compound in a local rather than systemic manner, for example, via injection of the compound directly into a solid tumor, often in a depot or sustained release formulation. Furthermore, one may administer the drug in a targeted drug delivery system, for example, in a liposome coated with tumor-specific antibody. In this way, the liposomes may be targeted to and taken up selectively by the tumor.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in any conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also. Pharmaceutical compositions which may also be used include hard gelatin capsules. The capsules or pills may be packaged into brown glass or plastic bottles to protect the active compound from light. The containers containing the active compound capsule formulation are preferably stored at controlled room temperature (15-30° C.).

For administration by inhalation, the compounds for use according to the present invention may be conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD cosolvent system. VPD is a solution of 3% w/v benzyl alcohol, 8% w/v of the nonpolar surfactant polysorbate 80, and 65% w/v polyethylene glycol 300, made up to volume in absolute ethanol. The VPD cosolvent system (VPD:D5W) consists of VPD diluted 1:1 with a 5% dextrose in water solution. This cosolvent system dissolves hydrophobic compounds well, and itself produces low toxicity upon systemic administration. Naturally, the proportions of such a cosolvent system may be varied considerably without destroying its solubility and toxicity characteristics. Furthermore, the identity of the cosolvent components may be varied: for example, other low-toxicity nonpolar surfactants may be used instead of polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the protein kinase-modulating compounds of the invention may be provided as physiologically acceptable salts wherein the claimed compound may form the negatively or the positively charged species. Examples of salts in which the compound forms the positively charged moiety include, without limitation, quaternary ammonium (defined elsewhere herein), salts such as the hydrochloride, sulfate, carbonate, lactate, tartrate, malate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), calcium hydroxide (Ca(OH)₂), etc.).

Pharmaceutical compositions suitable for use in the present invention include compositions wherein the active ingredients are contained in an amount sufficient to achieve the intended purpose, e.g., the modulation of protein kinase activity and/or the treatment or prevention of a protein kinase-related disorder.

More specifically, a therapeutically effective amount means an amount of compound effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the protein kinase activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ (both of which are discussed elsewhere herein) for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See, e.g., GOODMAN & GILMAN'S THE PHARMACOLOGICAL BASIS OF THERAPEUTICS, Ch. 3, 9^{th} ed., Ed. by Hardman, J., and Limbard, L., McGraw-Hill, New York City, 1996, p. 46.)

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of compounds of the present invention may range from approximately 2.5 mg/m² to 1500 mg/m² per day. Additional illustrative amounts range from 0.2-1000 mg/qid, 2-500 mg/qid, and 20-250 mg/qid.

In cases of local administration or selective uptake, the effective local concentration of the drug may not be related to plasma concentration, and other procedures known in the art may be employed to determine the correct dosage amount and interval.

The amount of a composition administered will, of course, be dependent on the subject being treated, the severity of the affliction, the manner of administration, the judgment of the prescribing physician, etc.

The compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

As mentioned above, the compounds and compositions of the invention will find utility in a broad range of diseases and conditions mediated by protein kinases, including diseases and conditions mediated by DNMT3b activity. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

The inventive compound can be used in combination with one or more other chemotherapeutic agents. The dosage of the inventive compounds may be adjusted for any drug-drug reaction. In one embodiment, the chemotherapeutic agent is selected from the group consisting of mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted ureas such as hydroxyurea; methylhydrazine derivatives, e.g., procarbazine; adrenocortical suppressants, e.g., mitotane, aminoglutethimide, hormone and hormone antagonists such as the adrenocorticosteriods (e.g., prednisone), progestins (e.g., hydroxyprogesterone caproate), estrogens (e.g., diethylstilbestrol), antiestrogens such as tamoxifen, androgens, e.g., testosterone propionate, and aromatase inhibitors, such as anastrozole, and AROMASIN (exemestane).

Examples of alkylating agents that the above method can be carried out in combination with include, without limitation, fluorouracil (5-FU) alone or in further combination with leukovorin; other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethylenimines and methylmelamines, e.g., altretamine, triethylenemelamine, trietylenephosphoramide, triethiylenethiophosphoramide and trimethylolomelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembichin, prednimustine and uracil mustard (used in the treatment of primary thrombocytosis, non-Hodgkin's lymphoma, Hodgkin's disease and ovarian cancer); and triazines, e.g., dacarbazine (used in the treatment of soft tissue sarcoma).

Examples of antimetabolite chemotherapeutic agents that the above method can be carried out in combination with include, without limitation, folic acid analogs, e.g., methotrexate (used in the treatment of acute lymphocytic leukemia, choriocarcinoma, mycosis fungoides, breast cancer, head and neck cancer and osteogenic sarcoma) and pteropterin; and the purine analogs such as mercaptopurine and thioguanine which find use in the treatment of acute granulocytic, acute lymphocytic and chronic granulocytic leukemias.

Examples of natural product-based chemotherapeutic agents that the above method can be carried out in combination with include, without limitation, the vinca alkaloids, e.g., vinblastine (used in the treatment of breast and testicular cancer), vincristine and vindesine; the epipodophyllotoxins, e.g., etoposide and teniposide, both of which are useful in the treatment of testicular cancer and Kaposi's sarcoma; the antibiotic chemotherapeutic agents, e.g., daunorubicin, doxorubicin, epirubicin, mitomycin (used to treat stomach, cervix, colon, breast, bladder and pancreatic cancer), dactinomycin, temozolomide, plicamycin, bleomycin (used in the treatment of skin, esophagus and genitourinary tract cancer); and the enzymatic chemotherapeutic agents such as L-asparaginase.

Examples of useful COX-II inhibitors include VIOXX, CELEBREX (celecoxib), valdecoxib, paracoxib, rofecoxib, and Cox 189.

Examples of useful matrix metalloproteinase inhibitors are described in WO 96/33172, WO 96/27583, European Patent Application No. 97304971.1, European Patent Application No. 99308617.2, WO 98/07697, WO 98/03516, WO 98/34918, WO 98/34915, WO 98/33768, WO 98/30566, European Patent Publication 606,046, European Patent Publication 931,788, WO 90/05719, WO 99/52910, WO 99/52889, WO 99/29667, PCT International Application No. PCT/IB98/01113, European Patent Application No. 99302232.1, Great Britain patent application number 9912961.1, U.S. Pat. No. 5,863,949, U.S. Pat. No. 5,861,510, and European Patent Publication 780,386, all of which are incorporated herein in their entireties by reference. Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and compounds selected from: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methylbenzyloxy)-benzenesulfonyl]-3-hydroxy-3-methyl-piperidine-2-carboxylic acid hydroxyamide; 3-[[(4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of these compounds.

Other anti-angiogenesis agents, other COX-II inhibitors and other MMP inhibitors, can also be used in the present invention.

An inventive compound can also be used with other signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example in WO 95/19970, WO 98/14451, WO 98/02434, and U.S. Pat. No. 5,747,498, and such substances can be used in the present invention as described herein.

EGFR-inhibiting agents include, but are not limited to, the monoclonal antibodies C225 and anti-EGFR 22Mab (ImClone Systems, Inc., New York, N.Y.), the compounds erlotinib (OSI Pharmaceuticals, Inc., Melville, N.Y.), ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.).

These and other EGFR-inhibiting agents can be used in the present invention. VEGF inhibitors, for example SU-5416 and SU-6668 (Sugen Inc., South San Francisco, Calif.), can also be combined with an inventive compound. VEGF inhibitors are described in, for example, WO 01/60814 A3, WO 99/24440, PCT International Application PCT/IB99/00797, WO 95/21613, WO 99/61422, U.S. Pat. No. 5,834,504, WO 01/60814, WO 98/50356, U.S. Pat. No. 5,883,113, U.S. Pat. No. 5,886,020, U.S. Pat. No. 5,792,783, WO 99/10349, WO 97/32856, WO 97/22596, WO 98/54093, WO 98/02438, WO 99/16755, and WO 98/02437, all of which are incorporated herein in their entireties by reference. Other examples of some specific VEGF inhibitors useful in the present invention are IM862 (Cytran Inc., Kirkland, Wash.); anti-VEGF monoclonal antibody of Genentech, Inc.; and angiozyme, a synthetic ribozyme from Ribozyme (Boulder, Colo.) and Chiron (Emeryville, Calif.). These and other VEGF inhibitors can be used in the present invention as described herein. Further, pErbB2 receptor inhibitors, such as GW-282974 (Glaxo Wellcome plc), and the monoclonal antibodies AR-209 (Aronex Pharmaceuticals Inc., The Woodlands, Tex.) and 2B-1 (Chiron), can furthermore be combined with an inventive compound, for example, those indicated in WO 98/02434, WO 99/35146, WO 99/35132, WO 98/02437, WO 97/13760, WO 95/19970, U.S. Pat. No. 5,587,458, and U.S. Pat. No. 5,877,305, which are all hereby incorporated herein in their entireties by reference. ErbB2 receptor inhibitors useful in the present invention are also described in U.S. Pat. No. 6,284,764, incorporated in its entirety herein by reference. The erbB2 receptor inhibitor compounds and substance described in the aforementioned PCT applications, U.S. patents, and U.S. provisional applications, as well as other compounds and substances that inhibit the erbB2 receptor, can be used with an inventive compound, in accordance with the present invention.

An inventive compound can also be used with other agents useful in treating cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, of U.S. Pat. No. 6,258,824 B1.

The above method can also be carried out in combination with radiation therapy, wherein the amount of an inventive compound in combination with the radiation therapy is effective in treating the above diseases. Techniques for administering radiation therapy are known in the art, and these techniques can be used in the combination therapy described herein. The administration of the compound of the invention in this combination therapy can be determined as described herein.

The invention will be further understood upon consideration of the following non-limiting Examples.

### EXAMPLES

### Protocols for DNMT3b Inhibition Assay

We use the DNMT3b Activity/Inhibition kit from Epigentek (cat# P-3007). Active DNMT3b enzyme is incubated with S-adenosylmethionine (SAM) in 96-well plates upon which unmethylated DNA has been immobilized onto the surface of each well. After incubation the reaction wells are washed and probed with a primary anti-methylcytosine antibody, which will bind to methylated DNA. Finally, a secondary antibody detects the primary antibody and creates a signal that is proportional to the amount of methylated DNA in the well. Uninhibited DNMT3b produces a well with high levels of methylated DNA (high signal), whereas inhibited DNMT3b produces a well with low levels of methylation (low signal). The protocol provided with the kit is slightly modified.

The final concentrations of reagents used in the DNMT3b assay are as follows:
100 μM SAM
2.2 μg/mL DNMT3b enzyme
Inhibitor concentrations typically range from 100 μM to 0.781 μM

General Protocol:

Test compounds are resuspended in 100% DMSO at 3 mM and serially diluted 1:2 in DMSO 8 times. A volume of 100 μL of 1× assay buffer is combined with 124 of 1 mM SAM solution and 4 μL of 66.7 μg/mL DNMT3b enzyme in the presence of 4 μL of test compound diluted in DMSO. Controls include enzyme only (no inhibitor, but containing 3.3% DMSO), no enzyme, and our best inhibitor. A volume of 30 μL of the mixed solution is then added to the substrate-coated wells in triplicate and incubated at 37° C. for 2 h. Reaction wells are then washed 3× with 150 μL of DNMT wash buffer. The primary antibody (anti-methylcytosine) is diluted 1:1000 in wash buffer, added to each well (50 μL) and incubated at rt for 1 h. Reaction wells are washed again. The secondary antibody is diluted 1:1000 in wash buffer, added to each well (50 μL) and incubated at rt for 30 min. The wells are washed for the last time. The developing solution is added to each well (100 μL) and incubated at rt for 4 min. The stop solution is added (50 μL) and the plate is immediately read on a plate reader at 450 nm absorbance.

Chemistry

Compounds of the invention may be made by one of ordinary skill in the chemical arts using conventional synthetic procedures, as well as by the general reaction schemes and examples described below (R₁, R₂, R₃, A, and X are given in Detailed Description of the Invention part).

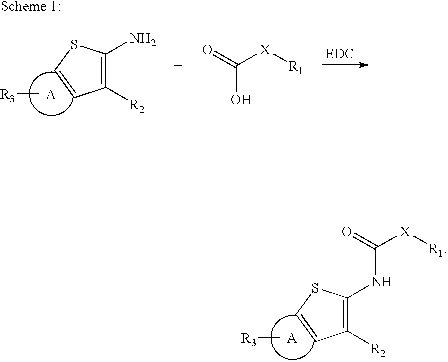

### Example 1

### S-2-(3-carbamoyl-6-methyl-4,5,6,7-tetrahydrobenzo[b]thiophen-2-ylamino)-2-oxoethyl 2-(1H-indol-3-yl)ethanethioate

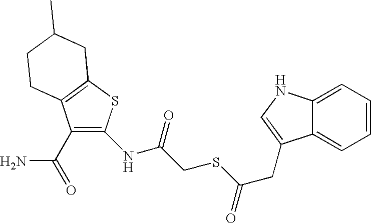

### Example 2

### 2-(2-(1H-indol-3-yloxy)acetamido)-4,5,6,7-tetrahydrobenzo[b]thiophene-3-carboxamide

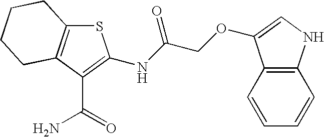

### Example 3

### 2-(2-(1H-indol-3-ylthio)acetamido)-4,5,6,7-tetrahydrobenzo[b]thiophene-3-carboxamide

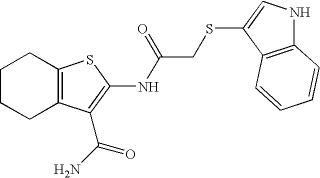

### Example 4

### 2-(2-(1H-indol-3-yloxy)propanamido)-4,5,6,7-tetrahydrobenzo[b]thiophene-3-carboxamide

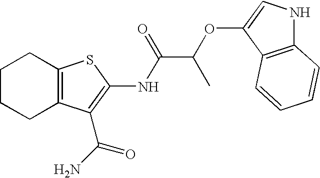

### Example 5

### 2-(2-(1H-indol-3-ylthio)propanamido)-4,5,6,7-tetrahydrobenzo[b]thiophene-3-carboxamide

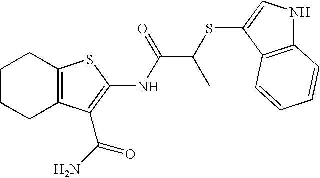

### Example 6

### 4-(2-(3-carbamoyl-4,5,6,7-tetrahydrobenzo[b]thiophen-2-ylamino)-2-oxoethoxy)pyrrolidine-2-carboxylic acid

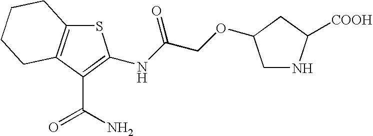

### Example 7

### 4-(2-(3-carbamoyl-4,5,6,7-tetrahydrobenzo[b]thiophen-2-ylamino)-2-oxoethylthio)pyrrolidine-2-carboxylic acid

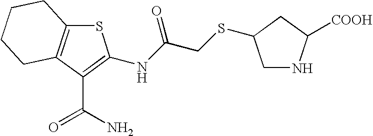

### Example 8

### 4-(1-(3-carbamoyl-4,5,6,7-tetrahydrobenzo[b]thiophen-2-ylamino)-1-oxopropan-2-yloxy)pyrrolidine-2-carboxylic acid

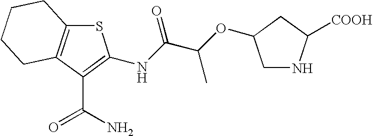

### Example 9

### 4-(1-(3-carbamoyl-4,5,6,7-tetrahydrobenzo[b]thiophen-2-ylamino)-1-oxopropan-2-ylthio)pyrrolidine-2-carboxylic acid

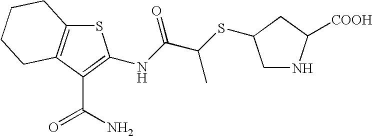

### Example 10

### 4-(2-oxo-2-(4,5,6,7-tetrahydrobenzo[b]thiophen-2-ylamino)ethylthio)pyrrolidine-2-carboxylic acid

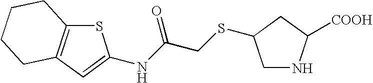

### Example 11

### 5-((1H-indol-3-yl)methyl)-N-(3-carbamoyl-6-methyl-4,5,6,7-tetrahydrobenzo[b]thiophen-2-yl)furan-2-carboxamide

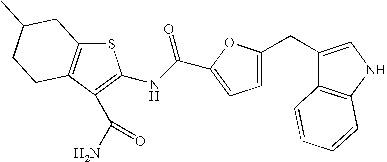

### Example 12

### N-(3-carbamoyl-4,5,6,7-tetrahydrobenzo[b]thiophen-2-yl)-5-nitrobenzo[b]thiophene-2-carboxamide

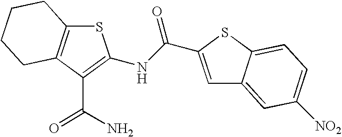

### Example 13

### N-(3-carbamoyl-4,5,6,7-tetrahydrobenzo[b]thiophen-2-yl)-5-nitrobenzofuran-2-carboxamide

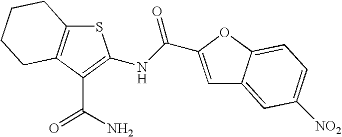

### Example 14

### N-(3-carbamoyl-4,5,6,7-tetrahydrobenzo[b]thiophen-2-yl)-5-nitro-1H-indole-2-carboxamide

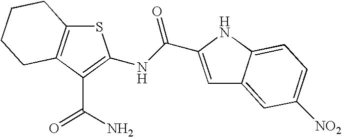

### Example 15

### 2-(2-(1H-indol-3-yloxy)acetamido)-5,6-dihydro-4H-cyclopenta[b]thiophene-3-carboxamide

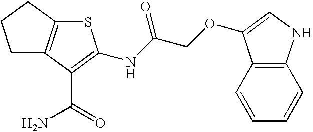

### Example 16

### 2-(2-(1H-indol-3-ylthio)acetamido)-5,6-dihydro-4H-cyclopenta[b]thiophene-3-carboxamide

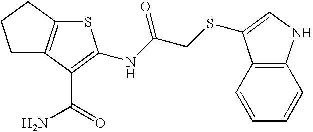

2-amino-5,6-dihydro-4H-cyclopenta[b]thiophene-3-carboxamide (91 mg, 0.5 mmol), 2-(1H-indol-3-ylthio)acetic acid (104 mg, 0.5 mmol) and EDC hydrochloride (144 mg, 0.75 mmol) were dissolved in 5 mL of DMF. Dark solution was stirred overnight. Reaction mixture was diluted with 300 mL of AcOEt. Solution was washed 3× with 1M NaOH, with brine, 3× with 1M HCl, with brine and dried over sodium sulfate. The solvent was removed to yield 143 mg of a brown crystalline solid. It was purified by MPLC (12 g silica column, gradient 0-20% AcOEt in DCM) to give yellow solid. It was boiled with small amount of methanol. After cooling solids were filtered off, washed with methanol and dried in vacuo to give pale yellow powder. Purity (HPLC): 100%. ESMS: ESMS: (M-H)⁻ 370.3 (calc. 370.1).

### Example 17

### 2-(2-(1H-indol-3-yloxy)propanamido)-5,6-dihydro-4H-cyclopenta[b]thiophene-3-carboxamide

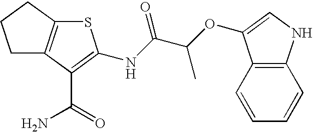

### Example 18

### 2-(2-(1H-indol-3-ylthio)propanamido)-5,6-dihydro-4H-cyclopenta[b]thiophene-3-carboxamide

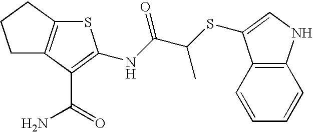

### Example 19

### 4-(2-(3-carbamoyl-5,6-dihydro-4H-cyclopenta[b]thiophen-2-ylamino)-2-oxoethoxy)pyrrolidine-2-carboxylic acid

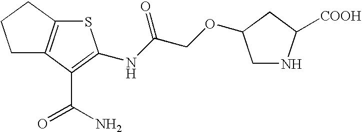

### Example 20

### 4-(2-(3-carbamoyl-5,6-dihydro-4H-cyclopenta[b]thiophen-2-ylamino)-2-oxoethylthio)pyrrolidine-2-carboxylic acid

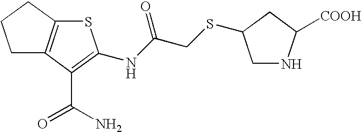

### Example 21

### 4-(1-(3-carbamoyl-5,6-dihydro-4H-cyclopenta[b]thiophen-2-ylamino)-1-oxopropan-2-yloxy)pyrrolidine-2-carboxylic acid

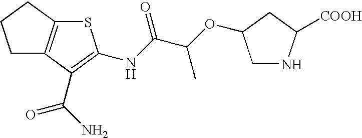

### Example 22

### 4-(1-(3-carbamoyl-5,6-dihydro-4H-cyclopenta[b]thiophen-2-ylamino)-1-oxopropan-2-ylthio)pyrrolidine-2-carboxylic acid

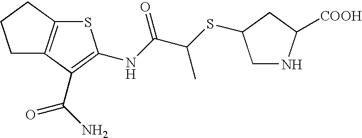

### Example 23

### 4-(2-(5,6-dihydro-4H-cyclopenta[b]thiophen-2-ylamino)-2-oxoethylthio)pyrrolidine-2-carboxylic acid

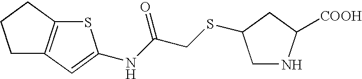

### Example 24

### 2-(2-(4-nitrophenoxy)acetamido)-5,6-dihydro-4H-cyclopenta[b]thiophene-3-carboxamide

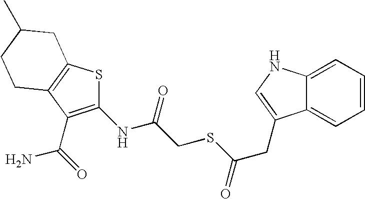

### Example 25

### 2-(2-(4-nitrophenylthio)acetamido)-5,6-dihydro-4H-cyclopenta[b]thiophene-3-carboxamide

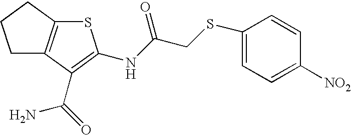

2-amino-5,6-dihydro-4H-cyclopenta[b]thiophene-3-carboxamide (182 mg, 1.0 mmol), 2-(4-nitrophenylthio)acetic acid (213 mg, 1.0 mmol) and EDC hydrochloride (288 mg, 1.5 mmol) were suspended in acetonitrile-DMF mixture (1:1, 20 mL). Pale brown suspension was stirred overnight. Suspension was poured into 200 mL of water, solids were filtered off, washed with water and air dried to give 315 mg of grey powder. It was suspended in 200 mL of boiling methanol and volume was reduced to ˜50 mL. After cooling solids were filtered off, washed with methanol and dried in vacuo to give grey powder. Purity (HPLC): 98%. ESMS: (M-H)⁻ 376.3 (calc. 376.0).

### Example 26

### 2-(2-(4-nitrophenoxy)propanamido)-5,6-dihydro-4H-cyclopenta[b]thiophene-3-carboxamide

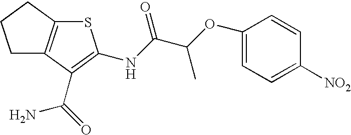

### Example 27

### 2-(2-(4-nitrophenylthio)propanamido)-5,6-dihydro-4H-cyclopenta[b]thiophene-3-carboxamide

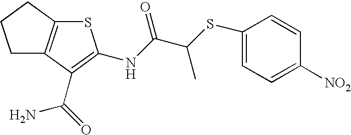

### Example 28

### 2-(2-(6-nitropyridin-3-ylthio)acetamido)-5,6-dihydro-4H-cyclopenta[b]thiophene-3-carboxamide

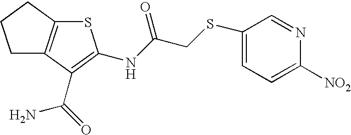

### Example 29

### 2-(2-(6-nitropyridin-3-ylthio)propanamido)-5,6-dihydro-4H-cyclopenta[b]thiophene-3-carboxamide

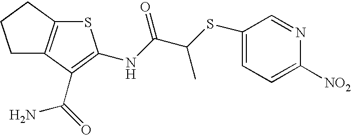

### Example 30

### 2-(2-(8-(phenethylamino)quinolin-4-yloxy)acetamido)-5,6-dihydro-4H-cyclopenta[b]thiophene-3-carboxamide

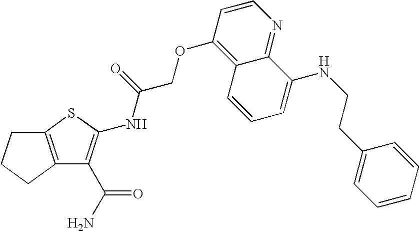

### Example 31

### 2-(2-(8-(phenethylamino)quinolin-4-ylthio)acetamido)-5,6-dihydro-4H-cyclopenta[b]thiophene-3-carboxamide

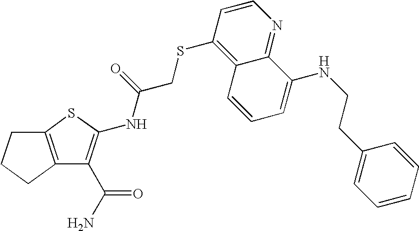

### Example 32

### 2-(2-(8-(phenethylamino)quinolin-4-yloxy)propanamido)-5,6-dihydro-4H-cyclopenta[b]thiophene-3-carboxamide

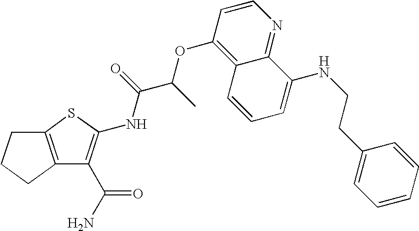

### Example 33

### 2-(2-(8-(phenethylamino)quinolin-4-ylthio)propanamido)-5,6-dihydro-4H-cyclopenta[b]thiophene-3-carboxamide

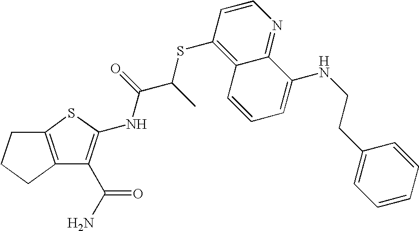

Representative EXAMPLES of the invention are set forth below in Tables 1, 2 and 3 below.

TABLE 1

| EX- | |
|---|---|
| AM- | |
| PLE | Structure |
| 1 | 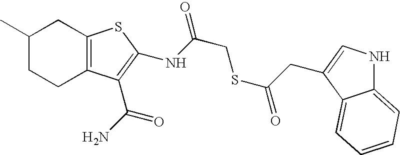 |
| 2 | 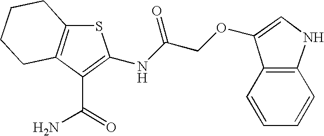 |
| 3 | 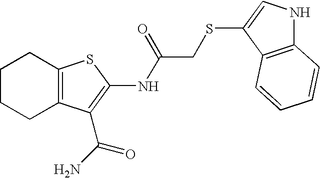 |
| 4 | 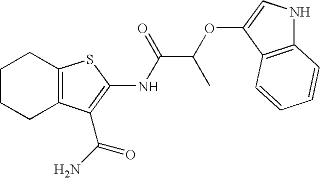 |
| 5 | 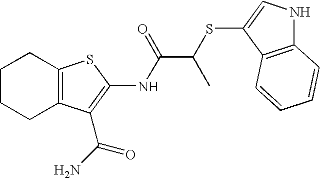 |
| 6 | 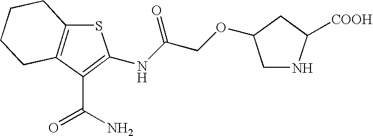 |
| 7 | 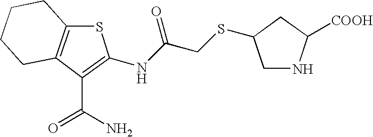 |
| 8 | 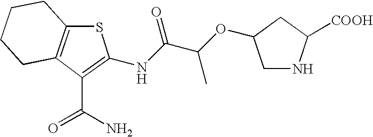 |
| 9 | 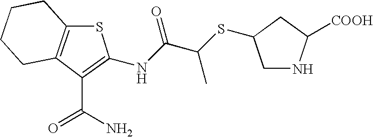 |
| 10 | 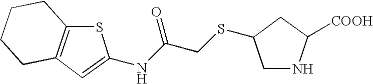 |

TABLE 2

| EX- | |
|---|---|
| AM- | |
| PLE | Structure |
| 11 | 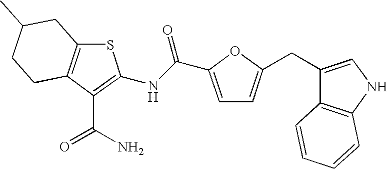 |
| 12 | 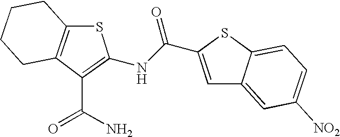 |
| 13 | 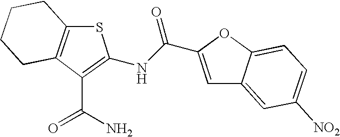 |
| 14 | 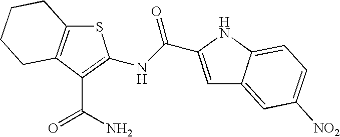 |

TABLE 3

| EX- | |
|---|---|
| AM- | |
| PLE | Structure |
| 15 | 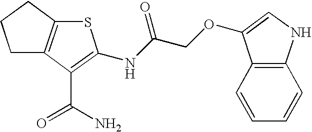 |
| 16 | 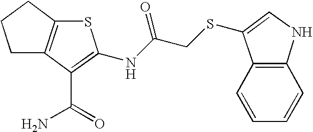 |
| 17 | 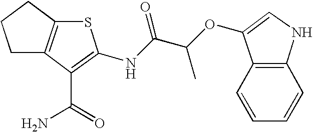 |
| 18 | 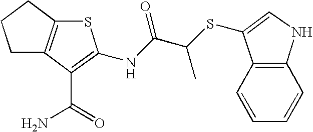 |
| 19 | 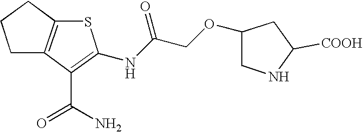 |
| 20 | 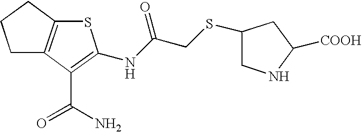 |
| 21 | 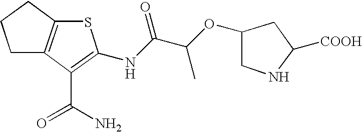 |
| 22 | 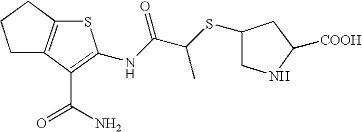 |
| 23 | 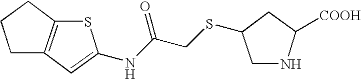 |
| 24 | 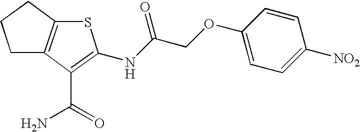 |
| 25 | 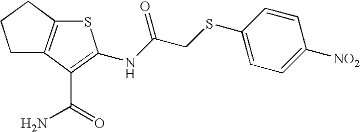 |
| 26 | 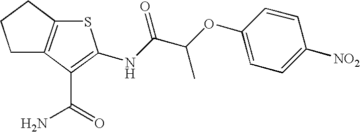 |
| 27 | 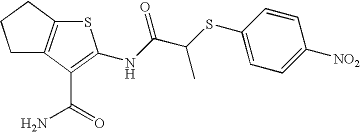 |
| 28 | 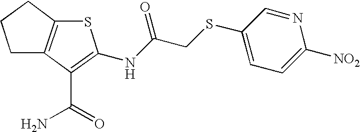 |
| 29 | 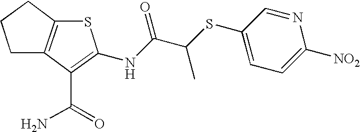 |
| 30 | 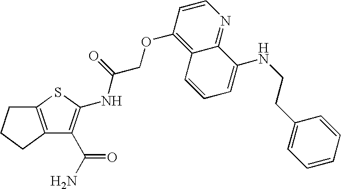 |
| 31 | 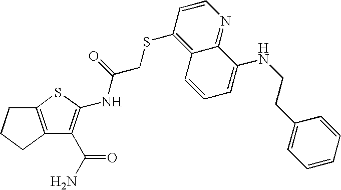 |
| 32 | 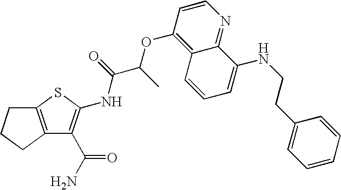 |
| 33 | 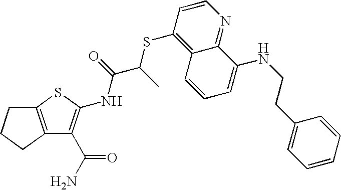 |

The compounds of the present invention include:
S-2-(3-carbamoyl-6-methyl-4,5,6,7-tetrahydrobenzo[b]thiophen-2-ylamino)-2-oxo ethyl 2-(1H-indol-3-yl)ethanethioate;
2-(2-(1H-indol-3-yloxy)acetamido)-4,5,6,7-tetrahydrobenzo[b]thiophene-3-carboxamide;
2-(2-(1H-indol-3-ylthio)acetamido)-4,5,6,7-tetrahydrobenzo[b]thiophene-3-carboxamide;
2-(2-(1H-indol-3-yloxy)propanamido)-4,5,6,7-tetrahydrobenzo[b]thiophene-3-carboxamide;
2-(2-(1H-indol-3-ylthio)propanamido)-4,5,6,7-tetrahydrobenzo[b]thiophene-3-carboxamide;
4-(2-(3-carbamoyl-4,5,6,7-tetrahydrobenzo[b]thiophen-2-ylamino)-2-oxo ethoxy)pyrrolidine-2-carboxylic acid;
4-(2-(3-carbamoyl-4,5,6,7-tetrahydrobenzo[b]thiophen-2-ylamino)-2-oxoethylthio)pyrrolidine-2-carboxylic acid;
4-(1-(3-carbamoyl-4,5,6,7-tetrahydrobenzo[b]thiophen-2-ylamino)-1-oxopropan-2-yloxy)pyrrolidine-2-carboxylic acid;
4-(1-(3-carbamoyl-4,5,6,7-tetrahydrobenzo[b]thiophen-2-ylamino)-1-oxopropan-2-ylthio)pyrrolidine-2-carboxylic acid;
4-(2-oxo-2-(4,5,6,7-tetrahydrobenzo[b]thiophen-2-ylamino)ethylthio)pyrrolidine-2-carboxylic acid;
5-((1H-indol-3-yl)methyl)-N-(3-carbamoyl-6-methyl-4,5,6,7-tetrahydrobenzo[b]thiophen-2-yl)furan-2-carboxamide;
N-(3-carbamoyl-4,5,6,7-tetrahydrobenzo[b]thiophen-2-yl)-5-nitrobenzo[b]thiophene-2-carboxamide;
N-(3-carbamoyl-4,5,6,7-tetrahydrobenzo[b]thiophen-2-yl)-5-nitrobenzofuran-2-carboxamide;
N-(3-carbamoyl-4,5,6,7-tetrahydrobenzo[b]thiophen-2-yl)-5-nitro-1H-indole-2-carboxamide;
2-(2-(1H-indol-3-yloxy)acetamido)-5,6-dihydro-4H-cyclopenta[b]thiophene-3-carboxamide;
2-(2-(1H-indol-3-ylthio)acetamido)-5,6-dihydro-4H-cyclopenta[b]thiophene-3-carboxamide;
2-(2-(1H-indol-3-yloxy)propanamido)-5,6-dihydro-4H-cyclopenta[b]thiophene-3-carboxamide;
2-(2-(1H-indol-3-ylthio)propanamido)-5,6-dihydro-4H-cyclopenta[b]thiophene-3-carboxamide;
4-(2-(3-carbamoyl-5,6-dihydro-4H-cyclopenta[b]thiophen-2-ylamino)-2-oxoethoxy)pyrrolidine-2-carboxylic acid;
4-(2-(3-carbamoyl-5,6-dihydro-4H-cyclopenta[b]thiophen-2-ylamino)-2-oxoethylthio)pyrrolidine-2-carboxylic acid;
4-(1-(3-carbamoyl-5,6-dihydro-4H-cyclopenta[b]thiophen-2-ylamino)-1-oxopropan-2-yloxy)pyrrolidine-2-carboxylic acid;
4-(1-(3-carbamoyl-5,6-dihydro-4H-cyclopenta[b]thiophen-2-ylamino)-1-oxopropan-2-ylthio)pyrrolidine-2-carboxylic acid;
4-(2-(5,6-dihydro-4H-cyclopenta[b]thiophen-2-ylamino)-2-oxoethylthio)pyrrolidine-2-carboxylic acid;
2-(2-(4-nitrophenoxy)acetamido)-5,6-dihydro-4H-cyclopenta[b]thiophene-3-carboxamide;
2-(2-(4-nitrophenylthio)acetamido)-5,6-dihydro-4H-cyclopenta[b]thiophene-3-carboxamide;
2-(2-(4-nitrophenoxy)propanamido)-5,6-dihydro-4H-cyclopenta[b]thiophene-3-carboxamide;
2-(2-(4-nitrophenylthio)propanamido)-5,6-dihydro-4H-cyclopenta[b]thiophene-3-carboxamide;
2-(2-(6-nitropyridin-3-ylthio)acetamido)-5,6-dihydro-4H-cyclopenta[b]thiophene-3-carboxamide;
2-(2-(6-nitropyridin-3-ylthio)propanamido)-5,6-dihydro-4H-cyclopenta[b]thiophene-3-carboxamide;
2-(2-(8-(phenethylamino)quinolin-4-yloxy)acetamido)-5,6-dihydro-4H-cyclopenta[b]thiophene-3-carboxamide;
2-(2-(8-(phenethylamino)quinolin-4-ylthio)acetamido)-5,6-dihydro-4H-cyclopenta[b]thiophene-3-carboxamide;
2-(2-(8-(phenethylamino)quinolin-4-yloxy)propanamido)-5,6-dihydro-4H-cyclopenta[b]thiophene-3-carboxamide;
2-(2-(8-(phenethylamino)quinolin-4-ylthio)propanamido)-5,6-dihydro-4H-cyclopenta[b]thiophene-3-carboxamide;

and pharmaceutically acceptable salts thereof.

Any U.S. patents, U.S. patent application publications, U.S. patent applications, foreign patents, foreign patent applications and non-patent publications referred to in this specification are incorporated herein by reference, in their entirety. From the foregoing it will be appreciated that, although specific embodiments of the invention have been described herein for purposes of illustration, various modifications may be made without deviating from the spirit and scope of the invention.

We claim:

1. A compound according to Formula (I): 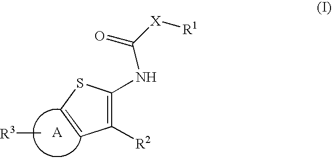
or a pharmaceutically acceptable salt thereof, wherein:
A is cyclopentenyl or cyclohexenyl;
X is —CH₂—O—, —CH₂—S—, —CH(CH₃)—O—, —CH(CH₃)—S—, -furanyl-CH₂—, or a direct bond; 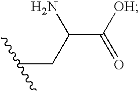
R¹ is aryl, heteroaryl, heterocyclyl, or each optionally substituted with 1-3 independent C₁₋₄alkyl, NO₂, COOH, or —NH(C₀₋₄alkyl)-aryl substituents;
R² is H, or —C(O)—NH₂; and
R³ is C₀₋₄alkyl;
provided that the compound is not:

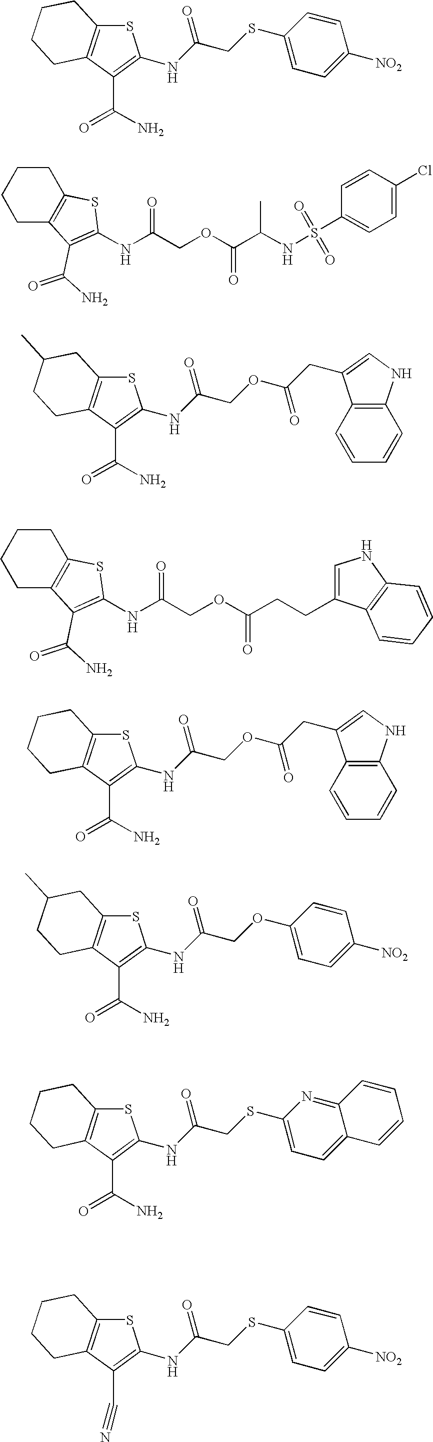

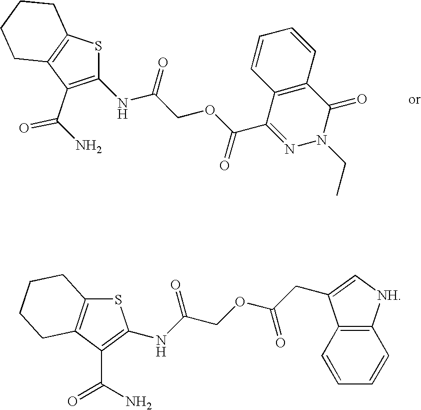

2. The compound according to claim 1, or a pharmaceutically acceptable salt thereof, wherein A is cyclopentenyl.

3. The compound according to claim 1, or a pharmaceutically acceptable salt thereof, wherein A is cyclopentenyl, and X is —CH₂—O—.

4. The compound according to claim 1, or a pharmaceutically acceptable salt thereof, wherein A is cyclopentenyl, X is —CH₂—O—, and R¹ is aryl optionally substituted with 1-3 independent C₁₋₄alkyl, NO₂, COOH, or —NH(C₀₋₄alkyl)-aryl substituents.

5. The compound according to claim 1, or a pharmaceutically acceptable salt thereof, wherein A is cyclopentenyl, X is —CH₂—O—, and R¹ is heterocyclyl optionally substituted with 1-3 independent C₁₋₄alkyl, NO₂, COOH, or —NH(C₀₋₄alkyl)-aryl substituents.

6. The compound according to claim 1, or a pharmaceutically acceptable salt thereof, wherein A is cyclopentenyl, X is —CH₂—O—, and R¹ is heteroaryl optionally substituted with 1-3 independent C₁₋₄alkyl, NO₂, COOH, or —NH(C₀₋₄alkyl)-aryl substituents.

7. The compound according to claim 1, or a pharmaceutically acceptable salt thereof, wherein A is cyclopentenyl, and X is —CH₂—S—.

8. The compound according to claim 1, or a pharmaceutically acceptable salt thereof, wherein A is cyclopentenyl, X is —CH₂—S—, and R¹ is aryl optionally substituted with 1-3 independent C₁₋₄alkyl, NO₂, COOH, or —NH(C₀₋₄alkyl)-aryl substituents.

9. The compound according to claim 1, or a pharmaceutically acceptable salt thereof, wherein A is cyclopentenyl, X is —CH₂—S—, and R¹ is heterocyclyl optionally substituted with 1-3 independent C₁₋₄alkyl, NO₂, COOH, or —NH(C₀₋₄alkyl)-aryl substituents.

10. The compound according to claim 1, or a pharmaceutically acceptable salt thereof, wherein A is cyclopentenyl, X is —CH₂—S—, and R¹ is heteroaryl optionally substituted with 1-3 independent C₁₋₄alkyl, NO₂, COOH, or —NH(C₀₋₄alkyl)-aryl substituents.

11. The compound according to claim 1, or a pharmaceutically acceptable salt thereof, wherein A is cyclopentenyl, and X is —CH(CH₃)—O—.

12. The compound according to claim 1, or a pharmaceutically acceptable salt thereof, wherein A is cyclopentenyl, X is —CH(CH₃)—O—, and R¹ is aryl optionally substituted with 1-3 independent C₁₋₄alkyl, NO₂, COOH, or —NH(C₀₋₄alkyl)-aryl substituents.

13. The compound according to claim 1, or a pharmaceutically acceptable salt thereof, wherein A is cyclopentenyl, X is —CH(CH₃)—O—, and R¹ is heterocyclyl optionally substituted with 1-3 independent C₁₋₄alkyl, NO₂, COOH, or —NH(C₀₋₄alkyl)-aryl substituents.

14. The compound according to claim 1, or a pharmaceutically acceptable salt thereof, wherein A is cyclopentenyl, X is —CH(CH₃)—O—, and R¹ is heteroaryl optionally substituted with 1-3 independent C₁₋₄alkyl, NO₂, COOH, or —NH(C₀₋₄alkyl)-aryl substituents.

15. The compound according to claim 1, or a pharmaceutically acceptable salt thereof, wherein A is cyclopentenyl, and X is —CH(CH₃)—S.

16. The compound according to claim 1, or a pharmaceutically acceptable salt thereof, wherein A is cyclopentenyl, X is —CH(CH₃)—S—, and R¹ is aryl optionally substituted with 1-3 independent C₁₋₄alkyl, NO₂, COOH, or —NH(C₀₋₄alkyl)-aryl substituents.

17. The compound according to claim 1, or a pharmaceutically acceptable salt thereof, wherein A is cyclopentenyl, X is —CH(CH₃)—S—, and R¹ is heterocyclyl optionally substituted with 1-3 independent C₁₋₄alkyl, NO₂, COOH, or —NH(C₀₋₄alkyl)-aryl substituents.

18. The compound according to claim 1, or a pharmaceutically acceptable salt thereof, wherein A is cyclopentenyl, X is —CH(CH₃)—S—, and R¹ is heteroaryl optionally substituted with 1-3 independent C₁₋₄alkyl, NO₂, COOH, or —NH(C₀₋₄alkyl)-aryl substituents.

19. The compound according to claim 1, or a pharmaceutically acceptable salt thereof, wherein A is cyclopentenyl, and X is -furanyl-CH₂—.

20. The compound according to claim 1, or a pharmaceutically acceptable salt thereof, wherein A is cyclopentenyl, X is -furanyl-CH₂—, and R¹ is aryl optionally substituted with 1-3 independent C₁₋₄alkyl, NO₂, COOH, or —NH(C₀₋₄alkyl)-aryl substituents.

21. The compound according to claim 1, or a pharmaceutically acceptable salt thereof, wherein A is cyclopentenyl, X is -furanyl-CH₂—, and R¹ is heterocyclyl optionally substituted with 1-3 independent C₁₋₄alkyl, NO₂, COOH, or —NH(C₀₋₄alkyl)-aryl substituents.

22. The compound according to claim 1, or a pharmaceutically acceptable salt thereof, wherein A is cyclopentenyl, X is -furanyl-CH₂—, and R¹ is heteroaryl optionally substituted with 1-3 independent C₁₋₄alkyl, NO₂, COOH, or —NH(C₀₋₄alkyl)-aryl substituents.

23. The compound according to claim 1, or a pharmaceutically acceptable salt thereof, wherein A is cyclopentenyl, and X is a direct bond.

24. The compound according to claim 1, or a pharmaceutically acceptable salt thereof, wherein A is cyclopentenyl, X is a direct bond, and R¹ is aryl optionally substituted with 1-3 independent C₁₋₄alkyl, NO₂, COOH, or —NH(C₀₋₄alkyl)-aryl substituents.

25. The compound according to claim 1, or a pharmaceutically acceptable salt thereof, wherein A is cyclopentenyl, X is a direct bond, and R¹ is heterocyclyl optionally substituted with 1-3 independent C₁₋₄alkyl, NO₂, COOH, or —NH(C₀₋₄alkyl)-aryl substituents.

26. The compound according to claim 1, or a pharmaceutically acceptable salt thereof, wherein A is cyclopentenyl, X is a direct bond, and R¹ is heteroaryl optionally substituted with 1-3 independent C₁₋₄alkyl, NO₂, COOH, or —NH(C₀₋₄alkyl)-aryl substituents.

27. The compound according to claim 1, or a pharmaceutically acceptable salt thereof, wherein A is cyclohexenyl.

28. The compound according to claim 1, or a pharmaceutically acceptable salt thereof, wherein A is cyclohexenyl, and X is —CH₂—O—.

29. The compound according to claim 1, or a pharmaceutically acceptable salt thereof, wherein A is cyclohexenyl, X is —CH₂—O—, and R¹ is aryl optionally substituted with 1-3 independent C₁₋₄alkyl, NO₂, COOH, or —NH(C₀₋₄alkyl)-aryl substituents.

30. The compound according to claim 1, or a pharmaceutically acceptable salt thereof, wherein A is cyclohexenyl, X is —CH₂—O—, and R¹ is heterocyclyl optionally substituted with 1-3 independent C₁₋₄alkyl, NO₂, COOH, or —NH(C₀₋₄alkyl)-aryl substituents.

31. The compound according to claim 1, or a pharmaceutically acceptable salt thereof, wherein A is cyclohexenyl, X is —CH₂—O—, and R¹ is heteroaryl optionally substituted with 1-3 independent C₁₋₄alkyl, NO₂, COOH, or —NH(C₀₋₄alkyl)-aryl substituents.

32. The compound according to claim 1, or a pharmaceutically acceptable salt thereof, wherein A is cyclohexenyl, and X is —CH₂—S—.

33. The compound according to claim 1, or a pharmaceutically acceptable salt thereof, wherein A is cyclohexenyl, X is —CH₂—S—, and R¹ is aryl optionally substituted with 1-3 independent C₁₋₄alkyl, NO₂, COOH, or —NH(C₀₋₄alkyl)-aryl substituents.

34. The compound according to claim 1, or a pharmaceutically acceptable salt thereof, wherein A is cyclohexenyl, X is —CH₂—S—, and R¹ is heterocyclyl optionally substituted with 1-3 independent C₁₋₄alkyl, NO₂, COOH, or —NH(C₀₋₄alkyl)-aryl substituents.

35. The compound according to claim 1, or a pharmaceutically acceptable salt thereof, wherein A is cyclohexenyl, X is —CH₂—S—, and R¹ is heteroaryl optionally substituted with 1-3 independent C₁₋₄alkyl, NO₂, COOH, or —NH(C₀₋₄alkyl)-aryl substituents.

36. The compound according to claim 1, or a pharmaceutically acceptable salt thereof, wherein A is cyclohexenyl, and X is —CH(CH₃)—O—.

37. The compound according to claim 1, or a pharmaceutically acceptable salt thereof, wherein A is cyclohexenyl, X is —CH(CH₃)—O—, and R¹ is aryl optionally substituted with 1-3 independent C₁₋₄alkyl, NO₂, COOH, or —NH(C₀₋₄alkyl)-aryl substituents.

38. The compound according to claim 1, or a pharmaceutically acceptable salt thereof, wherein A is cyclohexenyl, X is —CH(CH₃)—O—, and R¹ is heterocyclyl optionally substituted with 1-3 independent C₁₋₄alkyl, NO₂, COOH, or —NH(C₀₋₄alkyl)-aryl substituents.

39. The compound according to claim 1, or a pharmaceutically acceptable salt thereof, wherein A is cyclohexenyl, X is —CH(CH₃)—O—, and R¹ is heteroaryl optionally substituted with 1-3 independent C₁₋₄alkyl, NO₂, COOH, or —NH(C₀₋₄alkyl)-aryl substituents.

40. The compound according to claim 1, or a pharmaceutically acceptable salt thereof, wherein A is cyclohexenyl, and X is —CH(CH₃)—S—.

41. The compound according to claim 1, or a pharmaceutically acceptable salt thereof, wherein A is cyclohexenyl, X is —CH(CH₃)—S—, and R¹ is aryl optionally substituted with 1-3 independent C₁₋₄alkyl, NO₂, COOH, or —NH(C₀₋₄alkyl)-aryl substituents.

42. The compound according to claim 1, or a pharmaceutically acceptable salt thereof, wherein A is cyclohexenyl, X is —CH(CH₃)—S—, and R¹ is heterocyclyl optionally substituted with 1-3 independent C₁₋₄alkyl, NO₂, COOH, or —NH(C₀₋₄alkyl)-aryl substituents.

43. The compound according to claim 1, or a pharmaceutically acceptable salt thereof, wherein A is cyclohexenyl, X is —CH(CH₃)—S—, and R¹ is heteroaryl optionally substituted with 1-3 independent C₁₋₄alkyl, NO₂, COOH, or —NH(C₀₋₄alkyl)-aryl substituents.

44. The compound according to claim 1, or a pharmaceutically acceptable salt thereof, wherein A is cyclohexenyl, and X is -furanyl-CH₂—.

45. The compound according to claim 1, or a pharmaceutically acceptable salt thereof, wherein A is cyclohexenyl, X is -furanyl-CH₂—, and R¹ is aryl optionally substituted with 1-3 independent C₁₋₄alkyl, NO₂, COOH, or —NH(C₀₋₄alkyl)-aryl substituents.

46. The compound according to claim 1, or a pharmaceutically acceptable salt thereof, wherein A is cyclohexenyl, X is -furanyl-CH₂—, and R¹ is heterocyclyl optionally substituted with 1-3 independent C₁₋₄alkyl, NO₂, COOH, or —NH(C₀₋₄alkyl)-aryl substituents.

47. The compound according to claim 1, or a pharmaceutically acceptable salt thereof, wherein A is cyclohexenyl, X is -furanyl-CH₂—, and R¹ is heteroaryl optionally substituted with 1-3 independent C₁₋₄alkyl, NO₂, COOH, or —NH(C₀₋₄alkyl)-aryl substituents.

48. The compound according to claim 1, or a pharmaceutically acceptable salt thereof, wherein A is cyclohexenyl, and X is a direct bond.

49. The compound according to claim 1, or a pharmaceutically acceptable salt thereof, wherein A is cyclohexenyl, X is a direct bond, and R¹ is aryl optionally substituted with 1-3 independent C₁₋₄alkyl, NO₂, COOH, or —NH(C₀₋₄alkyl)-aryl substituents.

50. The compound according to claim 1, or a pharmaceutically acceptable salt thereof, wherein A is cyclohexenyl, X is a direct bond, and R¹ is heterocyclyl optionally substituted with 1-3 independent C₁₋₄alkyl, NO₂, COOH, or —NH(C₀₋₄alkyl)-aryl substituents.

51. The compound according to claim 1, or a pharmaceutically acceptable salt thereof, wherein A is cyclohexenyl, X is a direct bond, and R¹ is heteroaryl optionally substituted with 1-3 independent C₁₋₄alkyl, NO₂, COOH, or —NH(C₀₋₄alkyl)-aryl substituents.

52. The compound according to claim 1, consisting of 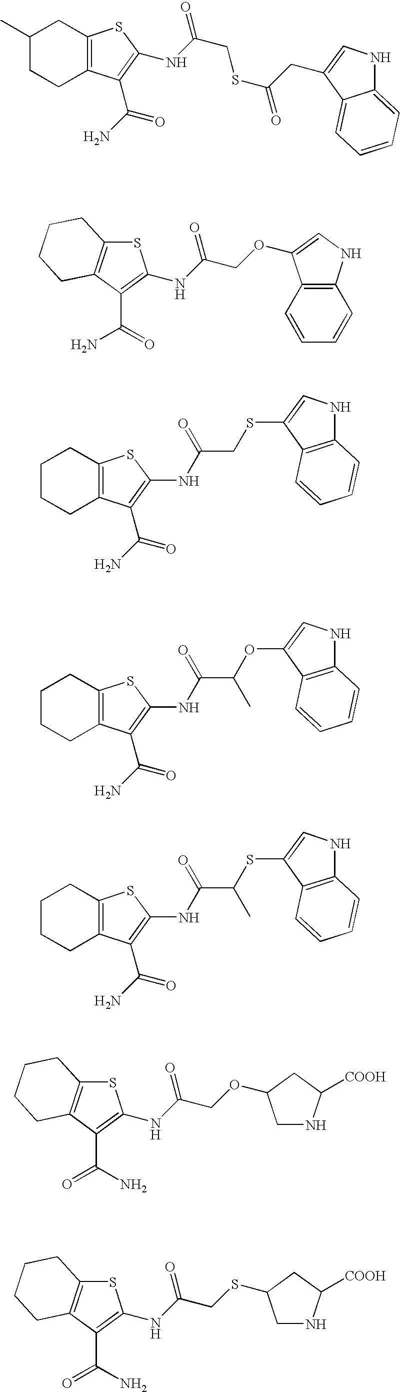 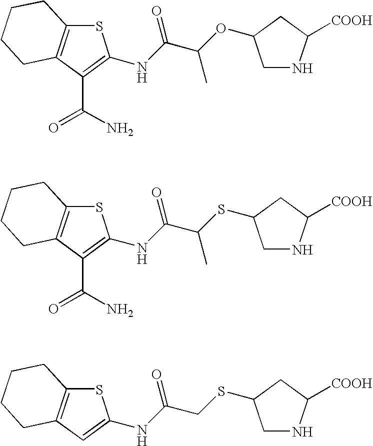
or a stereoisomer, or pharmaceutically acceptable salt thereof.

53. The compound according to claim 1, consisting of 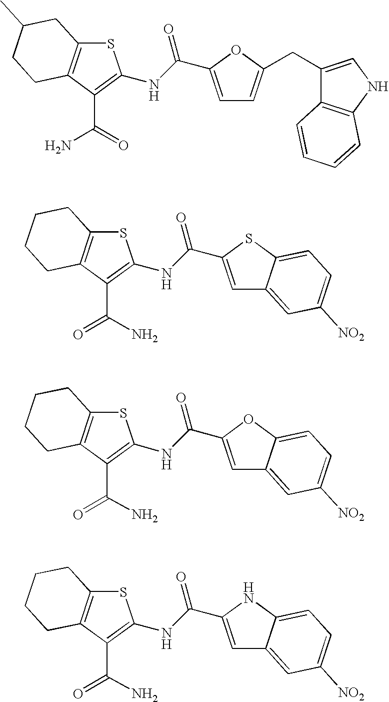
or a stereoisomer, or pharmaceutically acceptable salt thereof.

54. The compound according to claim 1, consisting of 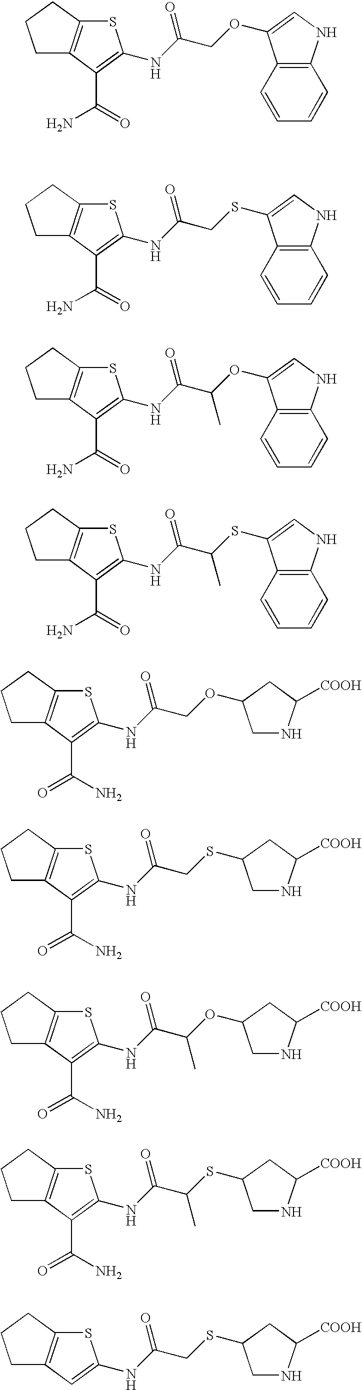 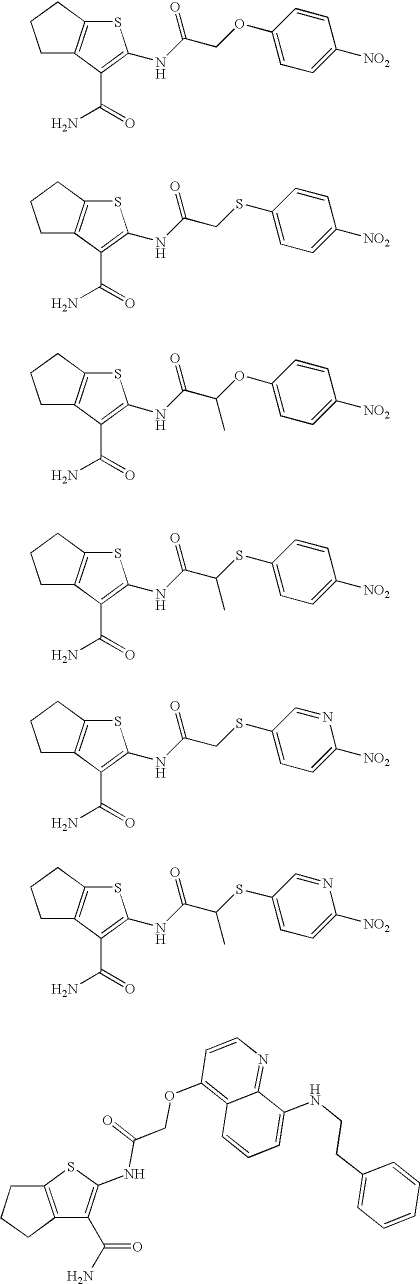 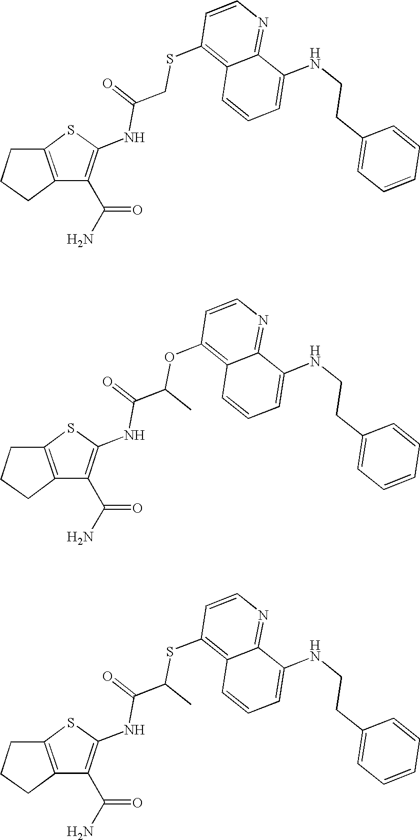
or a stereoisomer, or pharmaceutically acceptable salt thereof.

55. The compound according to claim 1, consisting of
S-2-(3-carbamoyl-6-methyl-4,5,6,7-tetrahydrobenzo[b]thiophen-2-ylamino)-2-oxoethyl 2-(1H-indol-3-yl)ethanethioate;
2-(2-(1H-indol-3-yloxy)acetamido)-4,5,6,7-tetrahydrobenzo[b]thiophene-3-carboxamide;
2-(2-(1H-indol-3-ylthio)acetamido)-4,5,6,7-tetrahydrobenzo[b]thiophene-3-carboxamide;
2-(2-(1H-indol-3-yloxy)propanamido)-4,5,6,7-tetrahydrobenzo[b]thiophene-3-carboxamide;
2-(2-(1H-indol-3-ylthio)propanamido)-4,5,6,7-tetrahydrobenzo[b]thiophene-3-carboxamide;
4-(2-(3-carbamoyl-4,5,6,7-tetrahydrobenzo[b]thiophen-2-ylamino)-2-oxoethoxy)pyrrolidine-2-carboxylic acid;
4-(2-(3-carbamoyl-4,5,6,7-tetrahydrobenzo[b]thiophen-2-ylamino)-2-oxoethylthio)pyrrolidine-2-carboxylic acid;
4-(1-(3-carbamoyl-4,5,6,7-tetrahydrobenzo[b]thiophen-2-ylamino)-1-oxopropan-2-yloxy)pyrrolidine-2-carboxylic acid;
4-(1-(3-carbamoyl-4,5,6,7-tetrahydrobenzo[b]thiophen-2-ylamino)-1-oxopropan-2-ylthio)pyrrolidine-2-carboxylic acid;
4-(2-oxo-2-(4,5,6,7-tetrahydrobenzo[b]thiophen-2-ylamino)ethylthio)pyrrolidine-2-carboxylic acid;
5-((1H-indol-3-yl)methyl)-N-(3-carbamoyl-6-methyl-4,5,6,7-tetrahydrobenzo[b]thiophen-2-yl)furan-2-carboxamide;
N-(3-carbamoyl-4,5,6,7-tetrahydrobenzo[b]thiophen-2-yl)-5-nitrobenzo[b]thiophene-2-carboxamide;
N-(3-carbamoyl-4,5,6,7-tetrahydrobenzo[b]thiophen-2-yl)-5-nitrobenzofuran-2-carboxamide;
N-(3-carbamoyl-4,5,6,7-tetrahydrobenzo[b]thiophen-2-yl)-5-nitro-1H-indole-2-carboxamide;
2-(2-(1H-indol-3-yloxy)acetamido)-5,6-dihydro-4H-cyclopenta[b]thiophene-3-carboxamide;
2-(2-(1H-indol-3-ylthio)acetamido)-5,6-dihydro-4H-cyclopenta[b]thiophene-3-carboxamide;
2-(2-(1H-indol-3-yloxy)propanamido)-5,6-dihydro-4H-cyclopenta[b]thiophene-3-carboxamide;
2-(2-(1H-indol-3-ylthio)propanamido)-5,6-dihydro-4H-cyclopenta[b]thiophene-3-carboxamide;
4-(2-(3-carbamoyl-5,6-dihydro-4H-cyclopenta[b]thiophen-2-ylamino)-2-oxoethoxy)pyrrolidine-2-carboxylic acid;
4-(2-(3-carbamoyl-5,6-dihydro-4H-cyclopenta[b]thiophen-2-ylamino)-2-oxoethylthio)pyrrolidine-2-carboxylic acid;
4-(1-(3-carbamoyl-5,6-dihydro-4H-cyclopenta[b]thiophen-2-ylamino)-1-oxopropan-2-yloxy)pyrrolidine-2-carboxylic acid;
4-(1-(3-carbamoyl-5,6-dihydro-4H-cyclopenta[b]thiophen-2-ylamino)-1-oxopropan-2-ylthio)pyrrolidine-2-carboxylic acid;
4-(2-(5,6-dihydro-4H-cyclopenta[b]thiophen-2-ylamino)-2-oxoethylthio)pyrrolidine-2-carboxylic acid;
2-(2-(4-nitrophenoxy)acetamido)-5,6-dihydro-4H-cyclopenta[b]thiophene-3-carboxamide;
2-(2-(4-nitrophenylthio)acetamido)-5,6-dihydro-4H-cyclopenta[b]thiophene-3-carboxamide;
2-(2-(4-nitrophenoxy)propanamido)-5,6-dihydro-4H-cyclopenta[b]thiophene-3-carboxamide;
2-(2-(4-nitrophenylthio)propanamido)-5,6-dihydro-4H-cyclopenta[b]thiophene-3-carboxamide;
2-(2-(6-nitropyridin-3-ylthio)acetamido)-5,6-dihydro-4H-cyclopenta[b]thiophene-3-carboxamide;
2-(2-(6-nitropyridin-3-ylthio)propanamido)-5,6-dihydro-4H-cyclopenta[b]thiophene-3-carboxamide;
2-(2-(8-(phenethylamino)quinolin-4-yloxy)acetamido)-5,6-dihydro-4H-cyclopenta[b]thiophene-3-carboxamide;
2-(2-(8-(phenethylamino)quinolin-4-ylthio)acetamido)-5,6-dihydro-4H-cyclopenta[b]thiophene-3-carboxamide;
2-(2-(8-(phenethylamino)quinolin-4-yloxy)propanamido)-5,6-dihydro-4H-cyclopenta[b]thiophene-3-carboxamide;
2-(2-(8-(phenethylamino)quinolin-4-ylthio)propanamido)-5,6-dihydro-4H-cyclopenta[b]thiophene-3-carboxamide;
or a stereoisomer, or a pharmaceutically acceptable salt thereof.

56. A method of treating cancer or hyperproliferative disorders by administering an effective amount of the compound according to claim 1.

57. The method of claim 56, wherein the cancer is of colon, breast, stomach, prostate, pancreas, or ovarian tissue.

58. A method of treating lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter, renal cell carcinoma, carcinoma of the renal pelvis, pediatric malignancy, neoplasms of the central nervous system, primary CNS lymphoma, spinal axis tumors, medulloblastoma, brain stem gliomas, pituitary adenomas, Barrett's esophagus, pre-malignant syndrome, neoplastic cutaneous disease, psoriasis, mycoses fungoides, benign prostatic hypertrophy, diabetic retinopathy, retinal ischemia, and retinal neovascularization, hepatic cirrhosis, angiogenesis, cardiovascular disease, atherosclerosis, immunological disease, autoimmune disease, or renal disease by administering to one in need of such treatment an effective amount of the compound according to claim 1.

59. A composition comprising a compound according to claim 1 and a pharmaceutically acceptable excipient.
